(19) United States
(12) Patent Application Publication
(10) Pub. No.: US 2025/0182504 A1
(43) Pub. Date: Jun. 5, 2025

(54) MORPHOLOGY BASED VERIFIABLE SCREENING

(71) Applicant: SCOPIO LABS LTD., Tel Aviv (IL)

(72) Inventors: Shahar KARNY, Hod HaSharon (IL); Ben LESHEM, Tel Aviv (IL); Erez NA'AMAN, Tel Aviv (IL); Eran SMALL, Yehud (IL); Itai HAYUT, Tel Aviv (IL)

(73) Assignee: SCOPIO LABS LTD., Tel Aviv (IL)

(21) Appl. No.: 18/833,694

(22) PCT Filed: Feb. 23, 2023
(86) PCT No.: PCT/IL2023/050190

§ 371 Date: Jul. 26, 2024

Related U.S. Application Data
(60) Provisional application No. 63/268,437, filed on Feb. 24, 2022.

(51) Int. Cl. G06V 20/69; G06V 10/74; G06V 10/764; G06V 10/94; G16H 10/60

(52) U.S. Cl. CPC G06V 20/698; G06V 10/74; G06V 10/764; G06V 10/945; G06V 20/693; G16H 10/60; G06V 2201/03

(57) ABSTRACT

A system comprises a plurality of user adjustable rules that allows a user to customize when a sample such as a peripheral blood smear is flagged for morphological review by a person and when an automated review of the sample is sufficient. The user adjustable rules allow a user to set rules that are appropriate for a healthcare provider to improve the work-flow and allows the user better control of the balance between automation of the sample review and manual review of the sample such as a peripheral blood smear. The user adjustable rules may comprise a set of values, such as thresholds or ranges for cell types, which may result in the sample being either flagged or not flagged for morphological review by a specialist such as a pathologist.

### RELATED APPLICATIONS

This application claims the benefit under 35 U.S.C. § 119 (e) of U.S. Provisional Patent Application No. 63/268,437, filed Feb. 24, 2022, which is incorporated, in its entirety, by this reference.

### BACKGROUND

Prior approaches to analyzing cells and cellular morphology from samples such as blood samples can be less than ideal in at least some respects. Prior approaches to analyzing the blood samples with microscopy include the manual review of microscope slides by a person who is qualified. This manual approach can be time consuming and somewhat limited because of the number of slides and cells on each side that are manually viewed. Also, the manual review of slides can be somewhat slower than would be ideal and somewhat limited by a lack of qualified personnel in at least some instances. Work in relation to the present disclosure suggests that the manual review of slides can be slower than would be ideal, which can lead to a delayed or incorrect diagnosis. For example, prior clinical standards for the review and analysis of blood samples such as a peripheral blood smear can be based on a compromise between what would be ideal and what can be achieved by a person manually reviewing slides. This can lead to a failure to detect rare cell types and morphology structures, which can lead to a flawed diagnosis in a least some instances. Also, the statistical sampling of prior approaches can be less than ideal because of the limited number of cells that can be analyzed, and in at least some instances diagnoses are made without statistical significance.

Although efforts have been made to digitize the analysis of samples such as a peripheral blood smear, the prior approaches may less than ideally reduce the manual aspect of slide review in at least some instance. Although approaches such as artificial intelligence have been proposed to review slides, these approaches may not appropriately detect potential problems with a sample or identification of cell types, which can result in further review by a person, often a person in the lab, in many instances. Also, some health care providers may be somewhat hesitant to rely on a fully automated system to analyze samples such as a peripheral blood smear in at least some instances. Therefore, there is a need to combine automated analysis of samples with manual review by a person. Work in relation to the present disclosure suggests that the prior approaches may have less than ideally balanced the tradeoff between automation and manual review of samples, which can result in a less than ideal work-flow in at least some instances. Also, the prior approaches may not have adequately addressed the allocation between automated and manual review, which can be specific to different health care providers and countries.

In light of the above, it would be beneficial to have improved methods, systems, apparatus and microscopes that ameliorate at least some of the aforementioned limitations of the prior approaches. For example, it would be helpful to allow users of at least partially automated systems to have more control over situations in which a sample is reviewed by a person and where the automated process for reviewing the sample is sufficient.

### SUMMARY

In some embodiments, the presently disclosed systems, methods and apparatus comprise a plurality of user adjustable rules that allow a user to customize when a sample such as a peripheral blood smear is flagged for morphological review by a person and when an automated review of the sample is sufficient. The user adjustable rules allow a user to set rules that are appropriate for a healthcare provider to improve the work-flow and allow the user better control of the balance between automated of the sample review and manual review of the sample such as a peripheral blood smear.

The user adjustable rules may comprise a set of values, such as thresholds or ranges for cell types, which may result in the sample being either flagged or not flagged for morphological review by a specialist such as a pathologist. In some embodiments, the sample is analyzed for a first plurality of cell types and compared with the set of user adjustable rules to determine whether to trigger a flag for morphological of the sample by the specialist such as a pathologist.

In some embodiments, the sample is analyzed for a second plurality of cell types, which is not compared with the set of user adjustable rules that would trigger human review of the morphology of the sample by a specialist such as a pathologist. For the second plurality of cell types, the numbers for each of the second plurality of cell types can be compared with rules that may be user adjustable in order to flag the results for review by a clinician, but not a morphological review of the sample. In some embodiments, the numbers for each of the second plurality of cell types can trigger a flag for further review of the results, but not a further review of the morphology of the sample. For the second plurality of cell types, values for the number of cells of each cell type can be reported to the patient record without triggering a flag for morphological review of the sample by a specialist such as a pathologist, even if the values are outside a normal range.

In some embodiments, the user interface is configured to receive a user input that switches a cell type between the first plurality of cell types and the second plurality of cell types, which can further improve the work-flow. In some embodiments, the user interface is configured for a user to identify a type of cell as belonging to either a first set of cell types corresponding to the first plurality of cell types or a second set of cell types corresponding to the second plurality of cell types, which can provide additional customization of the work-flow and potentially decrease manual review of samples by a specialist such as a pathologist.

In some embodiments, a computer implemented method of processing microscope image data comprises receiving microscope image data from a slide of a sample of the patient, in which the microscope image data comprises a plurality of cells comprising at least 100 white blood cells, at least 1000 red bloods cells, and platelet image data from an area of at least 50,000 μm². The microscope image data is processed with one or more classifiers to identify a plurality of cell types, in which the plurality of cell types comprises a first plurality of cell types and a second plurality of cell types. A number of cells is determined for each of the first plurality of cell types, and a number of cells is determined for each the second plurality of cell types. The number of cells of each of the first plurality of cell types is compared with a corresponding user adjustable rule, in which the corresponding user adjustable rule comprises one or more values for flagging or not flagging the sample for visual review by a specialist such as a pathologist. The sample is either flagged or not flagged for review in response to the comparing of the number of cells of said each of the first plurality of cell types with the corresponding user adjustable rule.

While the set of user adjustable rules for the first plurality of cell types can be configured in many ways, in some embodiments the set of rules comprises a plurality of rules for the first plurality of cell types. Alternatively or in combination, the set of rules may comprise a data structure, such as a look up table, which is configured to compare the number for each of the first plurality of cell types. Although reference is made to a plurality of user adjustable rules for each of the first plurality cell types, the set of rules may comprise a rule with sub-rules for determining whether to flag the sample for further morphological review by a specialist in response to the numbers of the first plurality of cell types.

While the cell types can be flagged in many ways, in some embodiments the sample is not flagged for further morphological review by a specialist in response to the number of cells of said each of the second plurality of cell types. In some embodiments, the numbers of cells of each of the first plurality of cell types and the second plurality of cell types is reported to a patient record, and when this occurs depends on whether the sample has been flagged or not. In some embodiments, the numbers of cells are automatically reported to the patient record in response to the sample not being flagged for further morphological review by a specialist such as a pathologist or other person trained in morphology. In some embodiments, the numbers of the cell types of the first and second pluralities of cell types are not reported to the patient record until the sample has been reviewed by a specialist such as a pathologist, and the reported values may be adjusted by the specialist.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows a diagram of an exemplary microscope, in accordance with some embodiments;

FIG. 2 shows a method of processing a sample, in accordance with some embodiments; and

FIG. 3 shows an exemplary computing system, in accordance with some embodiments; and

FIG. 4 shows an exemplary network architecture, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The presently disclosed systems, methods and apparatuses are well suited for combination with prior approaches to analyzing samples such as blood samples and blood smears. For example, the optical scanning apparatus may comprise one or more components of a conventional microscope with a sufficient numerical aperture, or a computational microscope as described in U.S. patent application Ser. No. 15/775,389, filed on Nov. 10, 2016, entitled “Computational microscopes and methods for generating an image under different illumination conditions,” published as US20190235224. The system may comprise one or more components of an autofocus system, for example as described in U.S. Pat. No. 10,705,326, entitled “Autofocus system for a computational microscope”. While the system may comprise any suitable user interface and data storage, in some embodiments, the system comprises one or more components for data storage and user interaction as described in U.S. Pat. No. 10,935,779, entitled “Digital microscope which operates as a server”. The system may comprise one or more components of an autoloader for loading slides, for example as described in U.S. patent application Ser. No. 16/875,665, filed on May 15, 2020, entitled “Multi/parallel scanner”. The system may comprise one or more components for selectively scanning areas of a sample, for example as described in U.S. patent application Ser. No. 16/875,721, filed on May 15, 2020, entitled “Accelerating digital microscopy scans using empty/dirty area detection,” published as US20200278530. The system may comprise a grid with a known pattern to facilitate image reconstruction, for example as described in U.S. Pat. No. 10,558,029, entitled “System for image reconstruction using a known pattern”. The system may comprise one or more classifiers as described in U.S. Ser. No. 17/755,356, entitled, “Method and apparatus for visualization of bone marrow cell populations”, published as US20220415480 on Dec. 29, 2022, which can be modified by one of ordinary skill in the art in accordance with the present disclosure. Each of the aforementioned patents and applications is incorporated herein by reference.

The presently disclosed system and method is well suited for use with automated methods of analyzing cellular morphology and classifiers, for example as described in PCT/IL2021/051329, filed on Nov. 9, 2021, entitled “FULL FIELD MORPHOLOGY-PRECISE QUANTIFICATION OF CELLULAR AND SUB-CELLULAR MORPHOLOGICAL EVENTS IN RED/WHITE BLOOD CELLS”, published as WO/2022/097155 on Dec. 1, 2022.

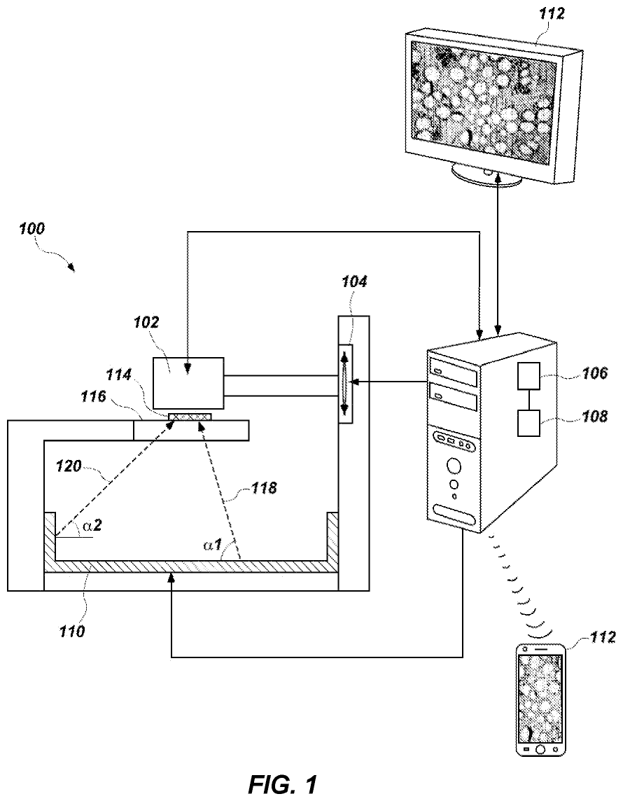

FIG. 1 is a diagrammatic representation of a microscope 100 consistent with the exemplary disclosed embodiments. The term “microscope” as used herein generally refers to any device or instrument for magnifying an object which is smaller than easily observable by the naked eye, e.g., capable of creating an image of an object for a user where the image is larger than the object. One type of microscope may be an “optical microscope” that uses light in combination with an optical system for magnifying an object. An optical microscope may be a simple microscope having one or more magnifying lens, or an optical microscope in which images are constructed from holograms with a digital holographic microscope, for example. Another type of microscope may be a “computational microscope” that comprises an image sensor and image-processing algorithms to enhance or magnify the object's size or other properties. The computational microscope may be a dedicated device or created by incorporating software and/or hardware with an existing optical microscope to produce high-resolution digital images. As shown in FIG. 1, microscope 100 comprises an image capture device 102, a focus actuator 104, a controller 106 connected to memory 108, an illumination assembly 110, and a user interface 112. An example usage of microscope 100 may be capturing images of a sample 114 mounted on a stage 116 located within the field-of-view (FOV) of image capture device 102, processing the captured images, and presenting on user interface 112 a magnified image of sample 114. Although reference is made to the presentation magnified images on a user interface, in some embodiments the processor is configured to automatically acquire the image data without displaying the magnified image of the sample 114 on the user interface, for example. Alternatively or in combination, the magnified image can be viewed by a user for morphological review of the sample, for example when the sample has been flagged for morphological review by a specialist such as a pathologist as described herein.

Image capture device 102 may be used to capture images of sample 114. In this specification, the term “image capture device” as used herein generally refers to a device that records the optical signals entering a lens as an image or a sequence of images. The optical signals may be in the near-infrared, infrared, visible, and ultraviolet spectrums. Examples of an image capture device comprise a CCD camera, a CMOS camera, a color camera, a photo sensor array, a video camera, a mobile phone equipped with a camera, a webcam, a preview camera, a microscope objective and detector, etc. Some embodiments may comprise only a single image capture device 102, while other embodiments may comprise two, three, or even four or more image capture devices 102. In some embodiments, image capture device 102 may be configured to capture images in a defined field-of-view (FOV). Also, when microscope 100 comprises several image capture devices 102, image capture devices 102 may have overlap areas in their respective FOVs. Image capture device 102 may have one or more image sensors (not shown in FIG. 1) for capturing image data of sample 114. In other embodiments, image capture device 102 may be configured to capture images at an image resolution higher than VGA, higher than 1 Megapixel, higher than 2 Megapixels, higher than 5 Megapixels, 10 Megapixels, higher than 12 Megapixels, higher than 15 Megapixels, or higher than 20 Megapixels. In addition, image capture device 102 may also be configured to have a pixel size smaller than 15 micrometers, smaller than 10 micrometers, smaller than 5 micrometers, smaller than 3 micrometers, or smaller than 1.6 micrometer.

In some embodiments, microscope 100 comprises focus actuator 104. The term “focus actuator” as used herein generally refers to any device capable of converting input signals into physical motion for adjusting the relative distance between sample 114 and image capture device 102. Various focus actuators may be used, including, for example, linear motors, electrostrictive actuators, electrostatic motors, capacitive motors, voice coil actuators, magnetostrictive actuators, etc. In some embodiments, focus actuator 104 may comprise an analog position feedback sensor and/or a digital position feedback element. Focus actuator 104 is configured to receive instructions from controller 106 in order to make light beams converge to form a clear and sharply defined image of sample 114. In the example illustrated in FIG. 1, focus actuator 104 may be configured to adjust the distance by moving image capture device 102.

However, in other embodiments, focus actuator 104 may be configured to adjust the distance by moving stage 116, or by moving both image capture device 102 and stage 116. Microscope 100 may also comprise controller 106 for controlling the operation of microscope 100 according to the disclosed embodiments. Controller 106 may comprise various types of devices for performing logic operations on one or more inputs of image data and other data according to stored or accessible software instructions providing desired functionality. For example, controller 106 may comprise a central processing unit (CPU), support circuits, digital signal processors, integrated circuits, cache memory, or any other types of devices for image processing and analysis such as graphic processing units (GPUs). The CPU may comprise any number of microcontrollers or microprocessors configured to process the imagery from the image sensors. For example, the CPU may comprise any type of single- or multi-core processor, mobile device microcontroller, etc. Various processors may be used, including, for example, processors available from manufacturers such as Intel®, AMD®, etc. and may comprise various architectures (e.g., x86 processor, ARM®, etc.). The support circuits may be any number of circuits generally well known in the art, including cache, power supply, clock and input-output circuits. Controller 106 may be at a remote location, such as a computing device communicatively coupled to microscope 100.

In some embodiments, controller 106 may be associated with memory 108 used for storing software that, when executed by controller 106, controls the operation of microscope 100. In addition, memory 108 may also store electronic data associated with operation of microscope 100 such as, for example, captured or generated images of sample 114. In one instance, memory 108 may be integrated into the controller 106. In another instance, memory 108 may be separated from the controller 106.

Specifically, memory 108 may refer to multiple structures or computer-readable storage mediums located at controller 106 or at a remote location, such as a cloud server. Memory 108 may comprise any number of random-access memories, read only memories, flash memories, disk drives, optical storage, tape storage, removable storage and other types of storage.

Microscope 100 may comprise illumination assembly 110. The term “illumination assembly” as used herein generally refers to any device or system capable of projecting light to illuminate sample 114.

Illumination assembly 110 may comprise any number of light sources, such as light emitting diodes (LEDs), LED array, lasers, and lamps configured to emit light, such as a halogen lamp, an incandescent lamp, or a sodium lamp. For example, illumination assembly 110 may comprise a Kohler illumination source. Illumination assembly 110 may be configured to emit polychromatic light. For instance, the polychromatic light may comprise white light.

In some embodiments, illumination assembly 110 may comprise only a single light source. Alternatively, illumination assembly 110 may comprise four, sixteen, or even more than a hundred light sources organized in an array or a matrix. In some embodiments, illumination assembly 110 may use one or more light sources located at a surface parallel to illuminate sample 114. In other embodiments, illumination assembly 110 may use one or more light sources located at a surface perpendicular or at an angle to sample 114.

In addition, illumination assembly 110 may be configured to illuminate sample 114 in a series of different illumination conditions. In one example, illumination assembly 110 may comprise a plurality of light sources arranged in different illumination angles, such as a two-dimensional arrangement of light sources. In this case, the different illumination conditions may comprise different illumination angles. For example, FIG. 1 depicts a beam 118 projected from a first illumination angle α1, and a beam 120 projected from a second illumination angle α2. In some embodiments, first illumination angle α1 and second illumination angle α2 may have the same value but opposite sign. In other embodiments, first illumination angle α1 may be separated from second illumination angle α2. However, both angles originate from points within the acceptance angle of the optics. In another example, illumination assembly 110 may comprise a plurality of light sources configured to emit light in different wavelengths. In this case, the different illumination conditions may comprise different wavelengths. For instance, each light source may be configured to emit light with a full width half maximum bandwidth of no more than 50 nm so as to emit substantially monochromatic light. In yet another example, illumination assembly 110 may be configured to use a number of light sources at predetermined times. In this case, the different illumination conditions may comprise different illumination patterns. For example, the light sources may be arranged to sequentially illuminate the sample at different angles to provide one or more of digital refocusing, aberration correction, or resolution enhancement. Accordingly and consistent with the present disclosure, the different illumination conditions may be selected from a group including different durations, different intensities, different positions, different illumination angles, different illumination patterns, different wavelengths, or any combination thereof.

Although reference is made to computational microscopy, the presently disclosed systems and methods are well suited for use with many types of microscopy and microscopes such as one or more of a high definition microscope, a digital microscope, a scanning digital microscope, a 3D microscope, a phase imaging microscope, a phase contrast microscope, a dark field microscope, a differential interference contrast microscope, a light-sheet microscope, a confocal microscope, a holographic microscope, or a fluorescence-based microscope.

In some embodiments, image capture device 102 may have an effective numerical aperture (“NA”) of at least 0.8, although any effective NA may be used. In some embodiments, the effective NA corresponds to a resolving power of the microscope that has the same resolving power as an objective lens with that NA under relevant illumination conditions. Image capture device 102 may also have an objective lens with a suitable NA to provide the effective NA, although the NA of the objective lens may be less than the effective NA of the microscope. For example, the imaging apparatus may comprise a computational microscope to reconstruct an image from a plurality of images captured with different illumination angles as described herein, in which the reconstructed image corresponds to an effective NA that is higher than the NA of the objective lens of the image capture device. In some embodiments with conventional microscopes, the NA of the microscope objective corresponds to the effective NA of the images. The lens may comprise any suitable lens such as an oil immersion lens or a non-oil immersion lens.

Consistent with disclosed embodiments, microscope 100 may comprise, be connected with, or in communication with (e.g., over a network or wirelessly, e.g., via Bluetooth) user interface 112. The term “user interface” as used herein generally refers to any device suitable for presenting data such as numerical results, microscope image data, or a magnified image of sample 114, or any device suitable for receiving inputs from one or more users of data related to microscope 100, such as remote users, for example. FIG. 1 illustrates two examples of user interface 112. The first example is a smartphone or a tablet wirelessly communicating with controller 106 over a Bluetooth, cellular connection or a Wi-Fi connection, directly or through a remote server. The second example is a PC display physically connected to controller 106. In some embodiments, user interface 112 may comprise user output devices, including, for example, a display, tactile device, speaker, etc. In other embodiments, user interface 112 may comprise user input devices, including, for example, a touchscreen, microphone, keyboard, pointer devices, cameras, knobs, buttons, etc. With such input devices, a user may be able to provide information inputs or commands to microscope 100 by typing instructions or information, providing voice commands, selecting menu options on a screen using buttons, pointers, or eye-tracking capabilities, or through any other suitable techniques for communicating information to microscope 100. User interface 112 may be connected (physically or wirelessly) with one or more processing devices, such as controller 106, to provide and receive information to or from a user and process that information. In some embodiments, such processing devices may execute instructions for responding to keyboard entries or menu selections, recognizing and interpreting touches and/or gestures made on a touchscreen, recognizing and tracking eye movements, receiving and interpreting voice commands, etc.

Microscope 100 may also comprise or be connected to stage 116. Stage 116 comprises any horizontal rigid surface where sample 114 may be mounted for examination. Stage 116 may comprise a mechanical connector for retaining a slide containing sample 114 in a fixed position. The mechanical connector may use one or more of the following: a mount, an attaching member, a holding arm, a clamp, a clip, an adjustable frame, a locking mechanism, a spring or any combination thereof. In some embodiments, stage 116 may comprise a translucent portion or an opening for allowing light to illuminate sample 114. For example, light transmitted from illumination assembly 110 may pass through sample 114 and towards image capture device 102. In some embodiments, stage 116 and/or sample 114 may be moved using motors or manual controls in the XY plane to enable imaging of multiple areas of the sample.

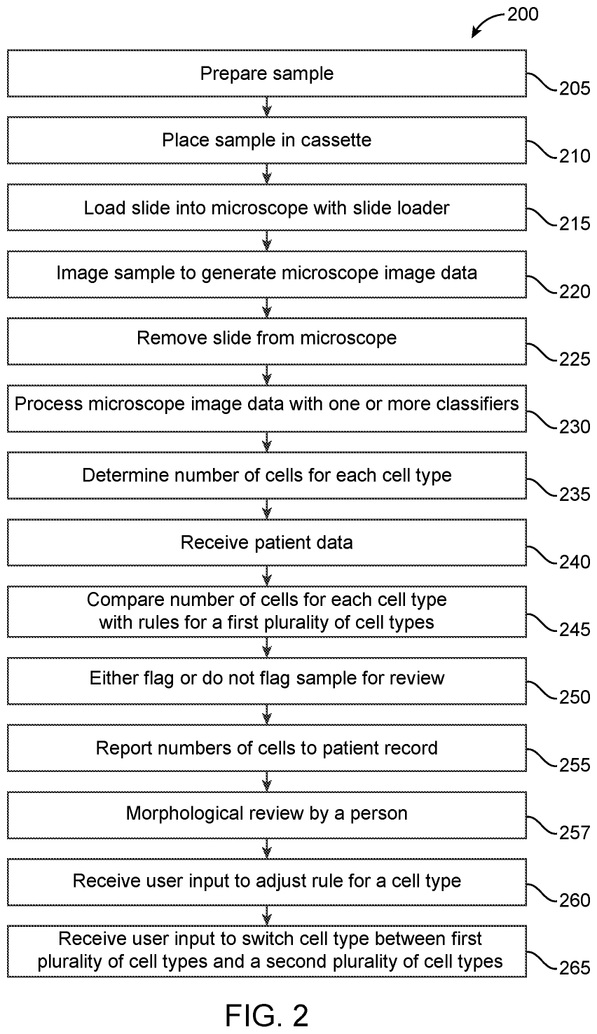

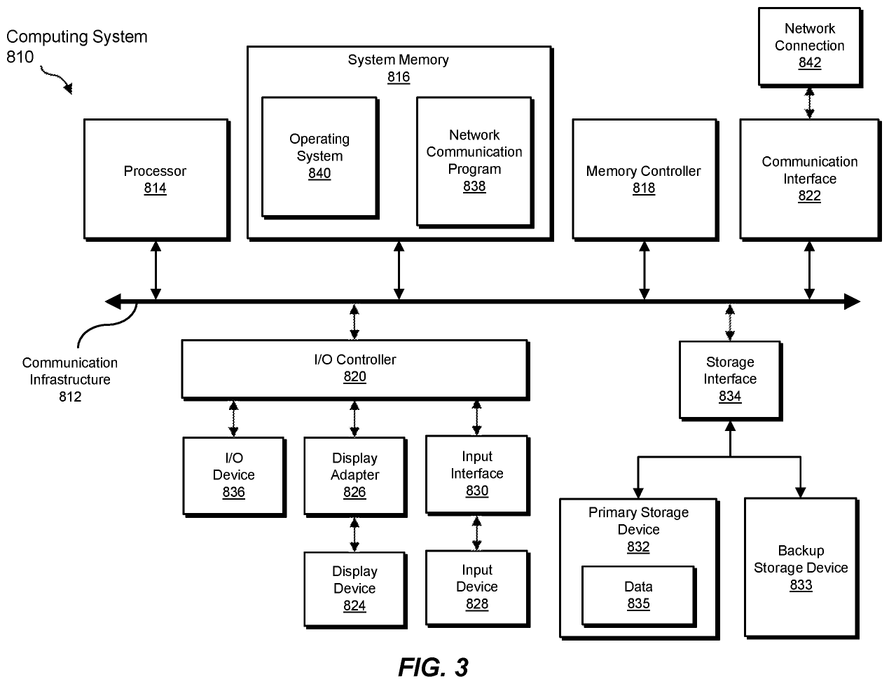

FIG. 2 is a flow diagram of an exemplary computer-implemented method 200 for processing microscope image data, in accordance with some embodiments. The steps shown in FIG. 2 may be performed by a microscope system, such as the system(s) illustrated in FIGS. 1, 3, and/or 4. In some embodiments, each of the steps shown in FIG. 2 may represent an algorithm whose structure includes and/or is represented by multiple sub-steps, examples of which will be provided in greater detail below.

As illustrated in FIG. 2, at step 205, the sample is prepared. The sample may comprise any suitable sample such as a peripheral blood smear on a slide, for example.

The slide may comprise a slide such as a glass or plastic slide having a substantially two dimensional surface on which a smear of the sample has been placed. Although reference is made to a smear of a sample on a slide, the sample may comprise a sample in a microfluidic chamber, for example.

At a step 210, the slide comprising the sample is placed in a cassette, although this step is optional as will be appreciated by one of ordinary skill in the art. For example, the slide may be manually placed in the microscope. Alternatively or in combination, the slides may be provided to the microscope with a coverslipping process and apparatus as described in PCT/IL2022/050565, filed on May 26, 2022, entitled “Systems and methods for coverslipping”, published as WO2022249191, on Dec. 1, 2022.

At a step 215, the slide is loaded into microscope with slide loader, although this step is optional as will be appreciated by one of ordinary skill in the art.

At a step 220, the sample is imaged to generate microscope image data and the microscope image data may comprise one or more images of the sample. In some embodiments, the microscope image data comprises a plurality of cells, in which the plurality of cells comprises at least 100 white blood cells, at least 1000 red bloods cells, and platelet image data from an area of at least 50,000 μm².

The microscope image data may comprise any suitable number of cells, and may comprise data from more than one slide for a patient, for example. In some embodiments, the sample comprises one or more of at least 200 white blood cells, at least 500 white blood cells or at least 1,000 white blood cells. In some embodiments, the sample comprises one or more of at least 2,000 red blood cells, at least 5,000 red blood cells or at least 10,000 white blood cells. In some embodiments, the sample comprises platelet image data from one or more of at least 100,000 μm², 200,000 μm², or at least 500,000 μm².

The one or more images may have an appropriate effective numerical aperture, such as at least one or more of 0.8, 0.9 or 1, for example. In some embodiments, the effective numerical aperture may be achieved by using a high numerical aperture lens in air. In some embodiments, a high numerical aperture may be achieved by using index matching material such as immersion oil or water. In some embodiments, a high numerical aperture may be achieved by using a lower numerical aperture index lens with computational methods that lead to a higher effective numerical aperture as described herein. In some embodiments, a high numerical aperture may be achieved by using a lens-less computational architecture, for example.

In some embodiments, the sample area may be divided up among multiple areas on a single slide or among a plurality of slides.

In some embodiments, the one or more images generated at step 220 may be generated with one or more imaging techniques. In some embodiments, the imaging techniques may include computational photography, computational imaging, digital holographic microscopy, computational microscopy, ptychography or Fourier ptychography, as discussed herein, for example, with respect to FIG. 1. In some embodiments, the image may be generated from a plurality of imaging conditions. For example, in some embodiments, the plurality of imaging conditions may include one or more of illuminating the sample at different illumination angles, illuminating the sample with different illumination patterns, or illuminating the sample with different wavelengths of light as described herein.

At a step 225, the slide is removed from the microscope. In some embodiments, the slide is removed with a slide loader and a new slide placed in the microscope, although this step is optional and may be done manually for example. Although reference is made to removing a slide from a slide loader, in some embodiments, the slide comprises a surface of a microfluidic chamber configured to view cells within the chamber, for example. In some embodiments, a plurality of slides is loaded and unloaded from a microscope with a slide loader and each of the plurality of slides is imaged with a microscope.

At a step 230, the microscope image data is processed. In some embodiments, the microscope image data is processed with one or more classifiers to identify a plurality of cell types, in which the plurality of cell types comprises a first plurality of cell types and a second plurality of cell types. The classifier may comprise any suitable classifier such one or more of a machine learning classifier, a neural network, or a convolutional neural network. A person of ordinary skill in the art can train a classifier in accordance with the present disclosure. The step 230 can be performed after, during, or partially overlapping with one or more of the steps of the method 200 described herein, such as partially over lapping with one or more of the step 220 of imaging the sample or the step 225 of removing the sample, for example.

The combination of the high resolution and field of view provided by the imaging discussed herein with the analysis and classification provides for analyzing and classifying a high number of cells at the same time. For example, an image of a sample at 100× magnification may include hundreds, thousands, tens of thousands, or even hundreds of thousands of cells that are input to the classifier. The one or more images that are processed with the classifier may comprise a plurality of images, a single image or several images that are combined, e.g., with scanning of a conventional microscope across several fields of view, or a plurality of images captured with a computational microscope and combined into an image with improved resolution as described herein. In some embodiments, the one or more images that are input to the classifier may comprises a scan of an area, which generates several images that are combined and input into the classifier, for example. The analysis and classification may be performed on an entire image or part of it, for example. Such analysis and classification can classify hundreds, thousands, tens of thousands, or hundreds of thousands of cells simultaneously from the image of the sample, for example. In some embodiments, at least 1000 cells, at least 10,000 cells, or at least 100,000 cells of a sample are imaged, processed and/or classified at the same time.

Sensitivity and specificity are measures of the ability of a test, such as a classifier, ability to correctly identify and classify a cell type in an image. In some embodiments, sensitivity refers to the classifier's ability to accurately designate a cell type in an image. In some embodiments, a highly sensitive test means that there are fewer false negative results, and thus fewer cases where the cell type is incorrectly identified. In some embodiments, a highly specific test means that there are few false positive results with respect to identifying a cell type.

Work in relation to the present disclosure suggests that image data from a slide from a sample of a patient comprising a plurality of cells comprising at least 100 white blood cells, at least 1000 red bloods cells, and platelet image data from an area of at least 50,000 μm² is well suited for the identification and classification of cell types in accordance with the present disclosure with high sensitivity and specificity, in which the first plurality of cell types comprises one or more of blasts, aberrant lymphocytes, intracellular micro-organisms, nucleated red blood cells (nRBCs), immature myeloids, smudge cells or schistocytes, and the second plurality of cell types comprises one or more of monocytes, toxic neutrophils, Large Granular Lymphocytes (LGLs), anisocytosis or platelet clumps. Additional numbers of cells and types from a sample can be identified and classified, which can increase the sensitivity and may be beneficial in accordance with some embodiments.

In some embodiments, the resolution of the image used in the classifier may be greater than the resolution of an image obtained in the imaging step. For example, imaging process, such as computational photography, computational imaging, computational microscopy, ptychography or Fourier ptychography may combine multiple images taken under different conditions into an image with a resolution higher than the constituent images used to generate the combined image. Similarly in some embodiments, the images may be acquired with an imaging sensor coupled to a microscope objective having a numerical aperture (NA). In some embodiments, after processing the images, such as through one or more of computational photography, computational imaging, computational microscopy, ptychography or Fourier ptychography and the resolution of the one or more images processed with the classifier corresponds to an effective NA greater than the NA of the microscope objective.

In some embodiments, the classification processing step 230 is performed with a plurality of separate processes, such as a plurality of threads, which allow the processing with the classifier to be performed more efficiently. In some embodiments, the processing classification step 230 comprises additional steps such as cell detection, cell segmentation, and cell classification, some of which may comprise sub-steps, or tasks, of the classification step 230. For example, the processing with the classifier may comprise sub-tasks that are performed in parallel, e.g. on the same or different processors. In some embodiments, the separate classifier processes are allocated to different processor resources such as separate cores of the processor or arranged into sub-tasks that run the processes in parallel on the same processor or different processors. The classifier processes may comprise sub-tasks that can be arranged in a que of sub-tasks to be performed on the same processor or different processor, and combinations thereof.

At a step 235, the number of cells for each cell type is determined. In some embodiments, a number of cells is determined for each of the first plurality of cell types and a number of cells is determined for each the second plurality of cell types.

At a step 240, patient data is received. The patient data may comprise any suitable patient data, such as one or more of an age or a sex of the patient. The patient data may comprise additional information such as results from a previous test, such as a blood count, for example a complete blood count (CBC) from a CBC analyzer. The patient data may comprise additional or alternative data, such as a weight or height of the patient, or medical history or possible illness, for example. The patient data can be received at any time or at different times, such as prior to imaging the sample or during any step or steps described herein. In some embodiments, the blood count data is received prior to imaging the sample and may affect the area of the sample that is imaged, for example.

At a step 245, the number of cells is compared for each cell type of the first plurality of cells with rules for flagging or not flagging the sample for further review. In some embodiments, the number of cells of said each of the first plurality of cell types is compared with a corresponding user adjustable rule, in which the corresponding user adjustable rule comprises one or more values for flagging or not flagging the sample for further review. In some embodiments, demographic data for a patient is received. The patient data may comprise one or more of an age or a sex of the patient, for example, and the comparison performed in response to the age of the patient.

In some embodiments, the first plurality of cell types comprises one or more of blasts, aberrant lymphocytes, intracellular micro-organisms, nucleated red blood cells (nRBCs), immature myeloids, smudge cells or schistocytes, and the second plurality of cell types comprises one or more of monocytes, toxic neutrophils, Large Granular Lymphocytes (LGLs), anisocytosis or platelet clumps. In some embodiments, each of the first plurality of cell types is compared to the user adjustable rule to either flag or not flag the sample for morphological review by a specialist such as a pathologist, and the second plurality of cell types is not compared to a rule to flag or not flag the sample for further review by a specialist such as a pathologist, although the numbers of the second plurality of cell types that are outside normal ranges may be flagged for consideration by a treating physician.

The adjustable rules for the cell types in the first plurality of cell types can be related to the age of the patient. In some embodiments, the age of the patient is greater than one month, and the first plurality of cell types comprises nRBCs. However, if the patient is less than one month old, nRBCs are typically more prevalent and the nRBCs are considered in the second plurality of cell types, in which elevated nRBCs would not flag the sample for further review by a specialist such as a pathologist, for example. Accordingly, the classification of a cell type as being among the first plurality of cell types, or the second plurality of cell types can depend on the age of the patient.

In some embodiments, the intracellular micro-organism comprises a pathogen such as malaria, for example.

In some embodiments, the immature myeloids comprise one or more of metamyelocytes, myelocytes, or promyelocytes, for example.

At a step 250, the sample is either flagged or not flagged for review by a specialist such as a pathologist. In some embodiments, the sample is either flagged or not flagged for further review in response to the comparing of the number of cells of said each of the first plurality of cell types with the corresponding user adjustable rule.

In some embodiments, the sample is not flagged for further review of the microscope image data in response to the number of cells of said each of the second plurality of cell types.

At a step 255, the number of cells for the plurality of cell types is reported to a patient record.

In some embodiments, at step 255 the method 200 may include generating a report of the sample. The values or characteristics in the report may be auto-populated according to the values from numbers of cell types.

At a step 257 morphological review of the sample is conducted by a person, such as a pathologist. In some embodiments, this review is triggered in response to the comparing of the numbers of cells of the first plurality of cell types to the user adjustable rules at step 245. Alternatively or in combination, this review is triggered in response to step 250 of either flagging or not flagging the sample for morphological review by a specialist such as a pathologist. In some embodiments, this review is conducted by the specialist viewing the images of the sample in response to the review flag being set at step 250. In some embodiments, the flag triggers a notification or alert or other indication for the morphological review by the specialist. In some embodiments, the numbers of the first plurality of cells are reported to the patient record after the review has been completed at step 257 if the sample has been flagged for review.

Although reference is made to review being conducted by a specialist in response to a flag for a specialist to review the sample, in some embodiments, the system comprises a decision support system configured for a user to review the numbers of each of the plurality cell types and corresponding images.

In some embodiments, the system presents supporting data from the analysis to aid in the decision or allow additional analysis or amending of the analysis, which can be helpful when step 257 is performed.

In some embodiments, the method may include using a decision support system for analysis by a user such as a remote user. The decision support system may compare the values of the detected objects with predefined values, which may be default values or determined by the user or center and can be adjusted based on data analysis and suggest if the values are within normal or abnormal range. The user may be given the option to override the suggestion and adjust the values. The decision support system may base its recommendation on properties as described herein, for example.

The decision support system may include graphic presentation of certain points of data. For example: presenting the scan with annotations around objects of interest detected, laying out the number/density of organisms by type, formation, context (e.g. inside or outside cells), color or any other characteristic. A decision support system suitable for incorporation in accordance with the present disclosure is described in PCT application no. PCT/IL2021/051329, published as WO2022/097155 on May 12, 2022, entitled “Full field morphology-precise quantification of cellular and sub-cellular morphological events in red/white blood cells”.

In some embodiments, the decision support system provides a portion of the one or more images corresponding to a location of a cell among the first plurality of cells detected with the classifier, which can facilitate review by the specialist. In some embodiments, the decision support system compares values of detected cells with reference values and indicates a comparison of the number of cells with a corresponding user adjustable rule. In some embodiments, the decision support system presents a portion of the one or more images with annotations around objects of interest and provides one or more of a number and density of the objects of interest by type, a formation of the objects of interest, a context of the objects of interest, an inside a cell context, an outside a cell context, or a color of the objects of interest.

At a step 260, a user input is received to adjust a rule for a cell type of the first plurality of cell types. In some embodiments, a user input is received to adjust at least one user adjustable rule and wherein the sample is flagged or not flagged in response to the user input. In some embodiments, the user interface is configured for a user to adjust one or more of a threshold value or a range of the user adjustable rule of said each of the first plurality of cell types.

In some embodiments, the user input is received prior to step 245, in which the number of cells is compared to the user adjustable rule, for example. In some embodiments, the sample is flagged or not flagged in response to a user adjusted rule for said each of the first plurality of cell types, for example.

While the rules can be configured and applied in many ways, in some embodiments, the number of each of the second plurality of cell types is not compared to a user adjustable rule to flag or not flag the sample for morphological review, e.g. by a specialist such as a pathologist, and the number of the second plurality of cell types is compared to a rule to flag or not flag the number for review by a clinician for said each of the second plurality of cell types. This may occur when the cell types of the second plurality of cell types is outside of a normal range, and morphological review by a specialist may not be helpful but flagging the result, e.g. the number of cells, is helpful for the treating physician.

In some embodiments, a user input is received to adjust the corresponding user adjustable rule for said each of the first plurality of cell types.

As many steps as appropriate of the method 200 can be automated. In some embodiments, the steps of receiving the microscope image data, processing the microscope image data, determining the number of cells, comparing the number of cells, and flagging or not flagging the sample are automated. In some embodiments, the method is fully automated from the step of loading and unloading the plurality of slides to the step of flagging or not flagging the sample. In some embodiments, if the sample is not flagged for further review such as morphological review by a person, the first plurality of cell types and the second plurality of cell types are reported to the patient record, and if the sample is flagged for further review by a person, the number of the first plurality of cell types is not reported to the patient record until a user input has been received indicating that the person has reviewed the morphology of the sample. Alternatively or in combination, if the sample has been flagged for review, in some embodiments the results for the second plurality of cell types are not reported to the patient record until the sample has been reviewed and a user input received indicating that the sample morphology has been reviewed by an appropriate person. In some embodiments, if the sample has been flagged for review, no results will be sent to the patient record until the sample has reviewed and an appropriate input received.

Although the order of the steps of the method 200 can be changed, in some embodiments the steps of receiving the microscope image data, processing the microscope image data, determining the number of cells, comparing the number of cells, and flagging or not flagging the sample are performed in sequence for a sample from a patient as shown in FIG. 2.

Referring again to FIG. 2, at a step 265, a user input is received to a switch cell type between first plurality of cell types and a second plurality of cell types. In some embodiments, the user adjustable rules are configured for the user to classify a cell type as being in either the first plurality of cells or the second plurality of cells.

In some embodiments, the first plurality of cell types initially comprises one or more of blasts, aberrant lymphocytes, intracellular micro-organisms, nucleated red blood cells (nRBCs), immature myeloids, smudge cells or schistocytes, and the second plurality of cell types initially comprises one or more of monocytes, toxic neutrophils, Large Granular Lymphocytes (LGLs), anisocytosis or platelet clumps. A user interface is configured for a user to move one or more of cell types from the first plurality of cell types to the second plurality of cell types. Alternatively or in combination, the user interface can be configured for a user to move one or more cell types from the second plurality of cell types to the first plurality of cell types, for example. In some embodiments, the user interface is configured to classify a cell type as belonging to either the first plurality of cell types or the second plurality of cell types, for example.

A person of ordinary skill in the art will recognize that method 200 of processing microscope image data can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. Also, some of the steps may be performed sequentially or at least partially simultaneously, for example. Further, some of the steps may be omitted, some of the steps repeated, and some of the steps may comprise substeps of other steps. Any of the steps may be automated or combined with an input from a user, such as a remote user or a user operating the system, for example.

The following example set of rules for a first plurality of cell types is provided values, thresholds and ranges that would trigger a manual, morphological review of the sample by a specialist in response to the number of cells of each of the first plurality of cell types in accordance with some embodiments:
Detecting 2 or more blasts;
Detecting 1 or more aberrant lymphocytes;
Detecting 1 or more intracellular microorganisms, such as Malaria;
Detecting 2 or more nRBCs in patients aged greater than 1 month (below that age, more nRBCs are to be expected);
Detecting >50 nRBCs for patients aged less than 1 month;
Detecting immature myeloids (metamyelocytes, myelocytes, promyelocytes) with a joint count of >10%;
Detecting a schistocyte level of ++ or +++; and
Detecting >200 smudge cells.

The following example is provided for examples for the second plurality of cell types that do not trigger a manual morphological review of the image in response to the cell count, in accordance with some embodiments:
Monocyte count;
Platelet clumps;
Detecting anisocytosis;
Toxic neutrophils; and
LGL count.

The rules for the first plurality of cell types may be adjusted as described herein. For example, the sample can be flagged for manual morphological review by a specialist if only 1 blast is detected, or if only 1 nRBC is detected in a patient aged older than 1 month, for example.

The rules can also be adjusted to allow a user to change a classification of a cell type as belonging to the first plurality of cell types or the second plurality of cell types as described herein, for example. Therefore, the above lists of the first plurality of cell types and the second plurality of cell types are provided merely as examples and can be modified in accordance with the present disclosure. By classifying cell types as belonging to a first plurality of cell types that can flag a sample for manual review by a specialist, or belong to a second plurality of cell types that do not flag the sample for the manual review, the instances of unnecessary manual reviews of samples by a specialist can be decreased and the overall work-flow improved.

A person of ordinary skill in the art can develop additional rules and change values as appropriate, for example with reference to clinically accepted standards such as the International Council for Standardization in Haematology (“ICSH”) guidelines.

Although reference is made to numbers of cells for each of the first plurality of cell types and the second plurality of cell types, these numbers may comprise any suitable parameter, such as an approximate value, a scored value such as + or ++, or any suitable metric as will be understood by one of ordinary skill in the art of haematology.

In accordance with some embodiments, Table 1 shows an example data structure such as a look up table comprising a set of rules that can be used to determine which cell types are used to trigger morphological review of the sample by a specialist and which cell types are not used to trigger the morphological review of the sample by the specialist. In some embodiments, a first category of cell types, e.g. category 1, comprises a first plurality of cell types and a second category of cell types, e.g. category 2, comprises a second plurality of cell types as described herein.

TABLE 1
Cell types, categories and rules.

| | | Rule/Flag value for | |
|---|---|---|---|
| | Cate- | manual review of | Rule/Flag value for |
| Cell Type | gory | sample | clinician review |
| blasts | 1 | Detecting 2 or more | Detecting 1 or more |
| aberrant | 1 | Detecting 1 or more | Detecting 1 or more |
| lymphocytes | | | |
| intracellular | 1 | Detecting 1 or more | Detecting 1 or more |
| microorganisms | | | |
| nRBCs | 1 | Detecting 2 or more in | Detecting 1 or more in |
| | | patients aged greater | patients aged greater |
| | | than 1 month (below | than 1 month (below |
| | | that age, more nRBCs | that age, more nRBCs |
| | | are to be expected) | are to be expected) |
| nRBCs | 1 | Detecting >50 for | Detecting >50 for |
| | | patients aged less | patients aged less |
| | | than 1 month | than 1 month |
| Immature | 1 | Joint count of >10% | Joint count of >10% |
| myeloids | | | |
| Schistocytes | 1 | level of ++ or +++ | level of ++ or +++ |
| Smudge cells | 1 | Detecting >200 | Detecting >200 |
| Monocytes | 2 | Not applicable | >8% |
| Platelet clumps | 2 | Not applicable | |
| Anisocytosis | 2 | Not applicable | +++ or higher |
| Toxic | 2 | Not applicable | >10% |
| neutrophils | | | |
| LGLs | 2 | Not applicable | >10% |

In some embodiments, the numbers of cells for the cell types identified as members of category 1 can trigger a flag for morphological review of the sample by a specialist and comprise the first plurality of cell types as described herein. In some embodiments, the numbers of cells for the cell types identified as members of category 2 cannot trigger a flag for morphological review of the sample by a specialist and comprise the second plurality of cell types as described herein.

In some embodiments, the rule comprises a first rule for determining whether to flag the sample for morphological review by a specialist and a second rule for flagging the number of cells for review by a clinician, and these rules can be applied independently to each of the plurality of cell types. For example, a first rule for flagging the sample for morphological review by a specialist may correspond to the presence of two or more blasts, and the second rule for flagging the number of cells for a clinician may comprise one or more blasts, for example. Alternatively or in combination, the first rule for flagging the sample for morphological review by a specialist may comprise a value of two or more blasts, and the second value for flagging the number of cells for review by a clinician may comprise one or more nRBCs, for example.

The user interface can be configured for a user to change the category of a cell type from the first category to the second category and vice versa, for example. In some embodiments, a data structure such as a table is shown to the user with the user interface and configured for the user to change the values in the data structure with user input. In some embodiments, the user interface is configured to change a cell type from category 1 to category 2, or from category 2 to category 1, for example. Once a cell type switches from category 2 to category 1, a rule for that cell type can be shown on the user interface and prepopulated with values, and the user interface configured to receive an input for the user accepting the rule. Alternatively or in combination, the user can input the rule directly into the user interface for example.

The data structure shown in Table 1 can be configured in many ways. In some embodiments, the flagging rule includes more than one cell type. In some embodiments, the category of the cell type comprises a category parameter, and the cell type comprises a cell type parameter associated with the corresponding user adjustable rule. The values of corresponding user adjustable rule shown in Table 1, may comprise values of a rule parameter, for example. The data structure can be arranged and may comprise a vector or matrix, for example. As many rules as appropriate can be included. For example, the data structure may comprise rules for flagging the numbers of a cell type in category 1 for further review by a clinician that do not trigger a morphological review of the sample by a specialist such as a pathologist as described herein. In some embodiments, the data structure such as the look up table is used as an input to a process that compares the number of cells for each of the plurality cell types to one or more rules as described herein.

Table 1 is shown as an example, and one of ordinary skill in the art will recognize many adaptations and variations in accordance with the present disclosure.

FIG. 3 is a block diagram of an example computing system 810 capable of implementing one or more of the embodiments described and/or illustrated herein. For example, all or a portion of computing system 810 may perform and/or be a means for performing, either alone or in combination with other elements, one or more of the steps described herein (such as one or more of the steps illustrated in FIG. 2). All or a portion of computing system 810 may also perform and/or be a means for performing any other steps, methods, or processes described and/or illustrated herein. All or a portion of computing system 810 may correspond to or otherwise be integrated with microscope 100 (e.g., one or more of controller 106, memory 108, and/or user interface 112).

Computing system 810 broadly represents any single or multi-processor computing device or system capable of executing computer-readable instructions. Examples of computing system 810 include, without limitation, workstations, laptops, client-side terminals, servers, distributed computing systems, handheld devices, or any other computing system or device. In its most basic configuration, computing system 810 may include at least one processor 814 and a system memory 816.

Processor 814 generally represents any type or form of physical processing unit (e.g., a hardware-implemented central processing unit) capable of processing data or interpreting and executing instructions. In certain embodiments, processor 814 may receive instructions from a software application or module. These instructions may cause processor 814 to perform the functions of one or more of the example embodiments described and/or illustrated herein.

In some embodiments, the classification is performed with a plurality of separate processes, such as a plurality of threads, which allow the processing with the classifier to be performed more efficiently on processor 814, which may comprise a single core processor, a multi core processor, or a plurality of processors, for example. In some embodiments, the separate classifier processes are allocated to different processor resources such as separate cores of the processor 814, or arranged into sub-tasks that run the processes in parallel on the same processor or different processors. The classifier processes may comprise sub-tasks that can be arranged in a que of sub-tasks to be performed on the same processor or different processor, and combinations thereof.

System memory 816 generally represents any type or form of volatile or non-volatile storage device or medium capable of storing data and/or other computer-readable instructions. Examples of system memory 816 include, without limitation, Random Access Memory (RAM), Read Only Memory (ROM), flash memory, or any other suitable memory device. Although not required, in certain embodiments computing system 810 may include both a volatile memory unit (such as, for example, system memory 816) and a non-volatile storage device (such as, for example, primary storage device 832, as described in detail below). In one example, one or more of steps from FIG. 2 may be computer instructions that may be loaded into system memory 816.

In some examples, system memory 816 may store and/or load an operating system 840 for execution by processor 814. In one example, operating system 840 may include and/or represent software that manages computer hardware and software resources and/or provides common services to computer programs and/or applications on computing system 810. Examples of operating system 840 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain embodiments, example computing system 810 may also include one or more components or elements in addition to processor 814 and system memory 816. For example, as illustrated in FIG. 3, computing system 810 may include a memory controller 818, an Input/Output (I/O) controller 820, and a communication interface 822, each of which may be interconnected via a communication infrastructure 812. Communication infrastructure 812 generally represents any type or form of infrastructure capable of facilitating communication between one or more components of a computing device. Examples of communication infrastructure 812 include, without limitation, a communication bus (such as an Industry Standard Architecture (ISA), Peripheral Component Interconnect (PCI), PCI Express (PCIe), or similar bus) and a network.

Memory controller 818 generally represents any type or form of device capable of handling memory or data or controlling communication between one or more components of computing system 810. For example, in certain embodiments memory controller 818 may control communication between processor 814, system memory 816, and I/O controller 820 via communication infrastructure 812.

I/O controller 820 generally represents any type or form of module capable of coordinating and/or controlling the input and output functions of a computing device. For example, in certain embodiments I/O controller 820 may control or facilitate transfer of data between one or more elements of computing system 810, such as processor 814, system memory 816, communication interface 822, display adapter 826, input interface 830, and storage interface 834.

As illustrated in FIG. 3, computing system 810 may also include at least one display device 824 (which may correspond to user interface 112) coupled to I/O controller 820 via a display adapter 826. Display device 824 generally represents any type or form of device capable of visually displaying information forwarded by display adapter 826. Similarly, display adapter 826 generally represents any type or form of device configured to forward graphics, text, and other data from communication infrastructure 812 (or from a frame buffer, as known in the art) for display on display device 824.

As illustrated in FIG. 3, example computing system 810 may also include at least one input device 828 (which may correspond to user interface 112) coupled to I/O controller 820 via an input interface 830. Input device 828 generally represents any type or form of input device capable of providing input, either computer or human generated, to example computing system 810. Examples of input device 828 include, without limitation, a keyboard, a pointing device, a speech recognition device, variations or combinations of one or more of the same, and/or any other input device.

Additionally or alternatively, example computing system 810 may include additional I/O devices. For example, example computing system 810 may include I/O device 836. In this example, I/O device 836 may include and/or represent a user interface that facilitates human interaction with computing system 810. Examples of I/O device 836 include, without limitation, a computer mouse, a keyboard, a monitor, a printer, a modem, a camera, a scanner, a microphone, a touchscreen device, variations or combinations of one or more of the same, and/or any other I/O device.

Communication interface 822 broadly represents any type or form of communication device or adapter capable of facilitating communication between example computing system 810 and one or more additional devices. For example, in certain embodiments communication interface 822 may facilitate communication between computing system 810 and a private or public network including additional computing systems. Examples of communication interface 822 include, without limitation, a wired network interface (such as a network interface card), a wireless network interface (such as a wireless network interface card), a modem, and any other suitable interface. In at least one embodiment, communication interface 822 may provide a direct connection to a remote server via a direct link to a network, such as the Internet. Communication interface 822 may also indirectly provide such a connection through, for example, a local area network (such as an Ethernet network), a personal area network, a telephone or cable network, a cellular telephone connection, a satellite data connection, or any other suitable connection.

In certain embodiments, communication interface 822 may also represent a host adapter configured to facilitate communication between computing system 810 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fibre Channel interface adapters, Ethernet adapters, or the like. Communication interface 822 may also allow computing system 810 to engage in distributed or remote computing. For example, communication interface 822 may receive instructions from a remote device or send instructions to a remote device for execution.

In some examples, system memory 816 may store and/or load a network communication program 838 for execution by processor 814. In one example, network communication program 838 may include and/or represent software that enables computing system 810 to establish a network connection 842 with another computing system (not illustrated in FIG. 3) and/or communicate with the other computing system by way of communication interface 822. In this example, network communication program 838 may direct the flow of outgoing traffic that is sent to the other computing system via network connection 842. Additionally or alternatively, network communication program 838 may direct the processing of incoming traffic that is received from the other computing system via network connection 842 in connection with processor 814.

Although not illustrated in this way in FIG. 3, network communication program 838 may alternatively be stored and/or loaded in communication interface 822. For example, network communication program 838 may include and/or represent at least a portion of software and/or firmware that is executed by a processor and/or Application Specific Integrated Circuit (ASIC) incorporated in communication interface 822.

As illustrated in FIG. 3, example computing system 810 may also include a primary storage device 832 and a backup storage device 833 coupled to communication infrastructure 812 via a storage interface 834. Storage devices 832 and 833 generally represent any type or form of storage device or medium capable of storing data and/or other computer-readable instructions. For example, storage devices 832 and 833 may be a magnetic disk drive (e.g., a so-called hard drive), a solid state drive, a floppy disk drive, a magnetic tape drive, an optical disk drive, a flash drive, or the like. Storage interface 834 generally represents any type or form of interface or device for transferring data between storage devices 832 and 833 and other components of computing system 810. In one example, data 835 (which may correspond to the captured images described herein) may be stored and/or loaded in primary storage device 832.

In certain embodiments, storage devices 832 and 833 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 832 and 833 may also include other similar structures or devices for allowing computer software, data, or other computer-readable instructions to be loaded into computing system 810. For example, storage devices 832 and 833 may be configured to read and write software, data, or other computer-readable information. Storage devices 832 and 833 may also be a part of computing system 810 or may be a separate device accessed through other interface systems.

Many other devices or subsystems may be connected to computing system 810. Conversely, all of the components and devices illustrated in FIG. 3 need not be present to practice the embodiments described and/or illustrated herein. The devices and subsystems referenced above may also be interconnected in different ways from that shown in FIG. 3. Computing system 810 may also employ any number of software, firmware, and/or hardware configurations. For example, one or more of the example embodiments disclosed herein may be encoded as a computer program (also referred to as computer software, software applications, computer-readable instructions, or computer control logic) on a computer-readable medium. The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 810. All or a portion of the computer program stored on the computer-readable medium may then be stored in system memory 816 and/or various portions of storage devices 832 and 833. When executed by processor 814, a computer program loaded into computing system 810 may cause processor 814 to perform and/or be a means for performing the functions of one or more of the example embodiments described and/or illustrated herein. Additionally or alternatively, one or more of the example embodiments described and/or illustrated herein may be implemented in firmware and/or hardware. For example, computing system 810 may be configured as an Application Specific Integrated Circuit (ASIC) adapted to implement one or more of the example embodiments disclosed herein.

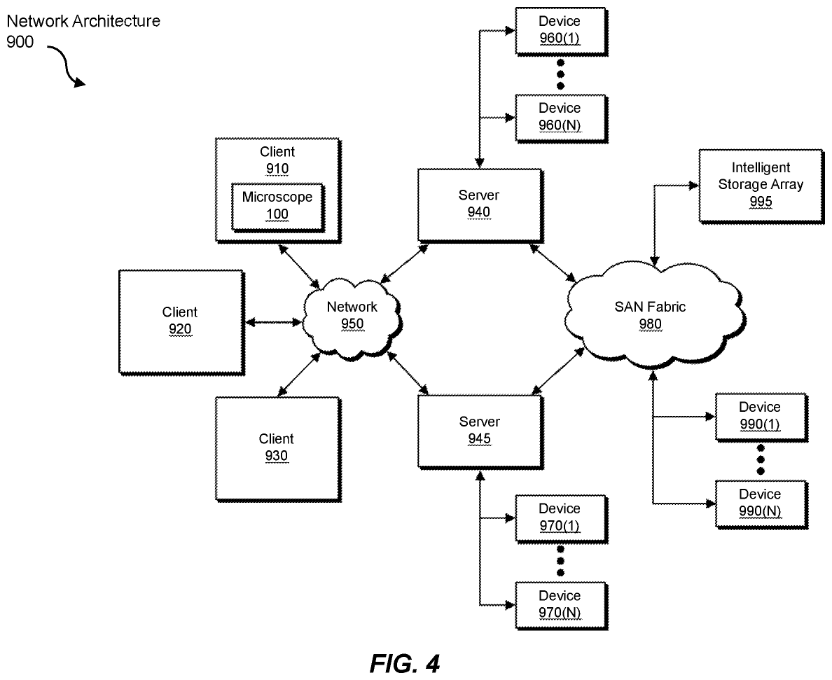

FIG. 4 is a block diagram of an example network architecture 900 in which client systems 910, 920, and 930 and servers 940 and 945 may be coupled to a network 950. As detailed above, all or a portion of network architecture 900 may perform and/or be a means for performing, either alone or in combination with other elements, one or more of the steps disclosed herein (such as one or more of the steps illustrated in FIG. 2). All or a portion of network architecture 900 may also be used to perform and/or be a means for performing other steps and features set forth in the instant disclosure.

Client systems 910, 920, and 930 generally represent any type or form of computing device or system, such as example computing system 810 in FIG. 3. Similarly, servers 940 and 945 generally represent computing devices or systems, such as application servers or database servers, configured to provide various database services and/or run certain software applications. Network 950 generally represents any telecommunication or computer network including, for example, an intranet, a WAN, a LAN, a PAN, or the Internet. In one example, client systems 910, 920, and/or 930 and/or servers 940 and/or 945 may include all or a portion of microscope 100 from FIG. 1.

As illustrated in FIG. 4, one or more storage devices 960(1)-(N) may be directly attached to server 940. Similarly, one or more storage devices 970(1)-(N) may be directly attached to server 945. Storage devices 960(1)-(N) and storage devices 970(1)-(N) generally represent any type or form of storage device or medium capable of storing data and/or other computer-readable instructions. In certain embodiments, storage devices 960(1)-(N) and storage devices 970(1)-(N) may represent Network-Attached Storage (NAS) devices configured to communicate with servers 940 and 945 using various protocols, such as Network File System (NFS), Server Message Block (SMB), or Common Internet File System (CIFS).

Servers 940 and 945 may also be connected to a Storage Area Network (SAN) fabric 980. SAN fabric 980 generally represents any type or form of computer network or architecture capable of facilitating communication between a plurality of storage devices. SAN fabric 980 may facilitate communication between servers 940 and 945 and a plurality of storage devices 990(1)-(N) and/or an intelligent storage array 995. SAN fabric 980 may also facilitate, via network 950 and servers 940 and 945, communication between client systems 910, 920, and 930 and storage devices 990(1)-(N) and/or intelligent storage array 995 in such a manner that devices 990(1)-(N) and array 995 appear as locally attached devices to client systems 910, 920, and 930. As with storage devices 960(1)-(N) and storage devices 970(1)-(N), storage devices 990(1)-(N) and intelligent storage array 995 generally represent any type or form of storage device or medium capable of storing data and/or other computer-readable instructions.

In certain embodiments, and with reference to example computing system 810 of FIG. 3, a communication interface, such as communication interface 822 in FIG. 3, may be used to provide connectivity between each client system 910, 920, and 930 and network 950. Client systems 910, 920, and 930 may be able to access information on server 940 or 945 using, for example, a web browser or other client software. Such software may allow client systems 910, 920, and 930 to access data hosted by server 940, server 945, storage devices 960(1)-(N), storage devices 970(1)-(N), storage devices 990(1)-(N), or intelligent storage array 995. Although FIG. 4 depicts the use of a network (such as the Internet) for exchanging data, the embodiments described and/or illustrated herein are not limited to the Internet or any particular network-based environment.

In at least one embodiment, all or a portion of one or more of the example embodiments disclosed herein may be encoded as a computer program and loaded onto and executed by server 940, server 945, storage devices 960(1)-(N), storage devices 970(1)-(N), storage devices 990(1)-(N), intelligent storage array 995, or any combination thereof. All or a portion of one or more of the example embodiments disclosed herein may also be encoded as a computer program, stored in server 940, run by server 945, and distributed to client systems 910, 920, and 930 over network 950.

As detailed above, computing system 810 and/or one or more components of network architecture 900 may perform and/or be a means for performing, either alone or in combination with other elements, one or more steps of method 200.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination unless indicated otherwise. In some embodiments, the term “or” is used exclusively to refer to items in the alternative only, for example in describing a work-flow of either flagging or not flagging the sample for further review.

As used herein, characters such as numerals refer to like elements.

As used herein, the terms “comprise” and “include” are interchangeable.

As used herein, the terms “in response to” and “based on” are interchangeable.

As used herein, the term resolution corresponds to the minimum distance at which an image of lines on a resolution target can be separated.

As used herein, mathematical symbols have their usual meaning as will be understood by one of ordinary skill in the art, unless indicated to the contrary. For example the symbols “<” and “>” have their normal meaning of less than and greater than, respectively.

The present disclosure includes the following numbered clauses.

Clause 1. A computer implemented method of processing microscope image data, the method comprising: receiving microscope image data from a slide from a sample of the patient, the microscope image data comprising a plurality of cells, the plurality of cells comprising at least 100 white blood cells, at least 1000 red bloods cells, and platelet image data from an area of at least 50,000 μm²; processing the microscope image data with one or more classifiers to identify a plurality of cell types, the plurality of cell types comprising a first plurality of cell types and a second plurality of cell types; determining a number of cells for each of the first plurality of cell types and a number of cells for each the second plurality of cell types; comparing the number of cells of said each of the first plurality of cell types with a corresponding user adjustable rule, the corresponding user adjustable rule comprising one or more values for flagging or not flagging the sample for further review; either flagging or not flagging the sample for further review in response to the comparing of the number of cells of said each of the first plurality of cell types with the corresponding user adjustable rule; and reporting the number of cells of said each of the first plurality of cell types and the number of cells of said each of the second plurality of cell types to a patient record.

Clause 2. The method of clause 1, wherein the sample is not flagged for further review of the microscope image data in response to the number of cells of said each of the second plurality of cell types.

Clause 3. The method of any of clauses 1 to 2, further comprising receiving a user input to adjust at least one user adjustable rule and wherein the sample is flagged or not flagged in response to the user input.

Clause 4. The method of any of clauses 1 to 3, wherein a user input is received to adjust the corresponding user adjustable rule for said each of the first plurality of cell types.

Clause 5. The method of any of clauses 1 to 4, wherein the sample is flagged or not flagged in response to a user adjusted rule for said each of the first plurality of cell types.

Clause 6. The method of any of clauses 1 to 5, wherein the number of said each of the second plurality of cell types is not compared to a user adjustable rule to flag or not flag the sample for morphological review and wherein the number is compared to a rule to flag or not flag for said each of the second plurality of cell types for review by a clinician.

Clause 7. The method of any of clauses 1 to 6, further comprising imaging the slide to generate the microscope image data.

Clause 8. The method of any of clauses 1 to 7, wherein the steps of receiving the microscope image data, processing the microscope image data, determining the number of cells, comparing the number of cells, and flagging or not flagging the sample are automated.

Clause 9. The method of any of clauses 1 to 8, wherein the steps of receiving the microscope image data, processing the microscope image data, determining the number of cells, comparing the number of cells, and flagging or not flagging the sample are performed in sequence.

Clause 10. The method of any of clauses 1 to 9, further comprising loading and unloading a plurality of slides from a microscope with a slide loader and wherein each of a plurality of slides is imaged with a microscope.

Clause 11. The method of any of clauses 1 to 10, wherein the method is fully automated from the step of loading and unloading the plurality of slides to the step of flagging or not flagging the sample.

Clause 12. The method of any of clauses 1 to 11, wherein if the sample is not flagged for further review by a person, the first plurality of cell types and the second plurality of cell types are reported to the patient record, and if the sample is flagged for further review by a person, the number of the first plurality of cell types is not reported to the patient record until a user input has been received indicating that the person has reviewed the sample.

Clause 13. The method of any of clauses 1 to 12, wherein the user adjustable rules are configured for the user to classify a cell type as being in either the first plurality of cells or the second plurality of cells and optionally wherein a user interface is configured for a user to switch a cell type between the first plurality of cell types and the second plurality of cell types.

Clause 14. The method of any of clauses 1 to 13, wherein a user interface is configured for a user to adjust one or more of a threshold value or a range of the user adjustable rule of said each of the first plurality of cell types.

Clause 15. The method of any of clauses 1 to 14, wherein the first plurality of cell types comprises one or more of blasts, aberrant lymphocytes, intracellular micro-organisms, nucleated red blood cells (nRBCs), immature myeloids, smudge cells or schistocytes, and the second plurality of cell types comprises one or more of monocytes, toxic neutrophils, Large Granular Lymphocytes (LGLs), anisocytosis or platelet clumps.

Clause 16. The method of any of clauses 1 to 15, further comprising receiving data for a patient, wherein the sample has been taken from the patient and wherein the patient data comprises one or more of an age or a sex of the patient.

Clause 17. The method of any of clauses 1 to 16, wherein the comparing is performed in response to the one or more of the age or the sex of the patient.

Clause 18. The method of any of clauses 1 to 17, wherein the age is greater than one month, and the first plurality of cell types comprises nRBCs.

Clause 19. The method of any of clauses 1 to 18, wherein the intracellular micro-organism comprises a pathogen and optionally wherein the pathogen comprises malaria.

Clause 20. The method of any of clauses 1 to 19, wherein the immature myeloids comprise one or more of metamyelocytes, myelocytes, or promyelocytes.

Clause 21. The method of any of clauses 1 to 20, wherein the sample comprises one or more of at least 200 white blood cells, at least 500 white blood cells or at least 1,000 white blood cells.

Clause 22. The method of any of clauses 1 to 21, wherein the sample comprises one or more of at least 2,000 red blood cells, at least 5,000 red blood cells or at least 10,000 white blood cells.

Clause 23. The method of any of clauses 1 to 22, wherein the sample comprises platelet image data from one or more of at least 100,000 μm², 200,000 μm², or at least 500,000 μm².

Clause 24. An apparatus comprising: a processor configured to perform the method of any of the preceding clauses.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A computer implemented method of processing microscope image data, the method comprising:
receiving microscope image data from a slide from a sample of the patient, the microscope image data comprising a plurality of cells, the plurality of cells comprising at least 100 white blood cells, at least 1000 red bloods cells, and platelet image data from an area of at least 50,000 μm²;
processing the microscope image data with one or more classifiers to identify a plurality of cell types, the plurality of cell types comprising a first plurality of cell types and a second plurality of cell types;
determining a number of cells for each of the first plurality of cell types and a number of cells for each the second plurality of cell types;
comparing the number of cells of said each of the first plurality of cell types with a corresponding user adjustable rule, the corresponding user adjustable rule comprising one or more values for flagging or not flagging the sample for further review;
either flagging or not flagging the sample for further review in response to the comparing of the number of cells of said each of the first plurality of cell types with the corresponding user adjustable rule; and
reporting the number of cells of said each of the first plurality of cell types and the number of cells of said each of the second plurality of cell types to a patient record.

2. The computer implemented method of claim 1, wherein the sample is not flagged for further review of the microscope image data in response to the number of cells of said each of the second plurality of cell types.

3. The computer implemented method of claim 1, further comprising receiving a user input to adjust at least one user adjustable rule and wherein the sample is flagged or not flagged in response to the user input.

4. The computer implemented method of claim 3, wherein a user input is received to adjust the corresponding user adjustable rule for said each of the first plurality of cell types.

5. The computer implemented method of claim 4, wherein the sample is flagged or not flagged in response to a user adjusted rule for said each of the first plurality of cell types.

6. The computer implemented method of claim 1, wherein the number of said each of the second plurality of cell types is not compared to a user adjustable rule to flag or not flag the sample for morphological review and wherein the number is compared to a rule to flag or not flag for said each of the second plurality of cell types for review by a clinician.

7. The computer implemented method of claim 1, further comprising imaging the slide to generate the microscope image data.

8. The computer implemented method of claim 1, wherein the steps of receiving the microscope image data, processing the microscope image data, determining the number of cells, comparing the number of cells, and flagging or not flagging the sample are automated.

9. The computer implemented method of claim 1, wherein the steps of receiving the microscope image data, processing the microscope image data, determining the number of cells, comparing the number of cells, and flagging or not flagging the sample are performed in sequence.

10. The computer implemented method of claim 1, further comprising loading and unloading a plurality of slides from a microscope with a slide loader and wherein each of a plurality of slides is imaged with a microscope.

11. The computer implemented method of claim 10, wherein the method is fully automated from the step of loading and unloading the plurality of slides to the step of flagging or not flagging the sample.

12. The computer implemented method of claim 11, wherein if the sample is not flagged for further review by a person, the first plurality of cell types and the second plurality of cell types are reported to the patient record, and if the sample is flagged for further review by a person, the number of the first plurality of cell types is not reported to the patient record until a user input has been received indicating that the person has reviewed the sample.

13. The computer implemented method of claim 1, wherein the user adjustable rules are configured for the user to classify a cell type as being in either the first plurality of cells or the second plurality of cells and optionally wherein a user interface is configured for a user to switch a cell type between the first plurality of cell types and the second plurality of cell types.

14. The computer implemented method of claim 1, wherein a user interface is configured for a user to adjust one or more of a threshold value or a range of the user adjustable rule of said each of the first plurality of cell types.

15. The computer implemented method of claim 1, wherein the first plurality of cell types comprises one or more of blasts, aberrant lymphocytes, intracellular micro-organisms, nucleated red blood cells (nRBCs), immature myeloids, smudge cells or schistocytes, and the second plurality of cell types comprises one or more of monocytes, toxic neutrophils, Large Granular Lymphocytes (LGLs), anisocytosis or platelet clumps.

16. The computer implemented method of claim 15, further comprising receiving data for a patient, wherein the sample has been taken from the patient and wherein the patient data comprises one or more of an age or a sex of the patient.

17. The computer implemented method of claim 16, wherein the comparing is performed in response to the one or more of the age or the sex of the patient.

18. The computer implemented method of claim 17, wherein the age is greater than one month, and the first plurality of cell types comprises nRBCs.

19. The computer implemented method of claim 15, wherein the intracellular micro-organism comprises a pathogen and optionally wherein the pathogen comprises malaria.

20. The computer implemented method of claim 15, wherein the immature myeloids comprise one or more of metamyelocytes, myelocytes, or promyelocytes.

21.-24. (canceled)
